(19) United States
(12) Patent Application Publication
(10) Pub. No.: US 2025/0302906 A1
(43) Pub. Date: Oct. 2, 2025

(54) SLEEP AID COMPOSITIONS

(71) Applicant: Melaleuca, Inc., Idaho Falls, ID (US)

(72) Inventors: Nasser A. Fredj, Idaho Falls, ID (US); Luping Yan, Ammon, ID (US)

(21) Appl. No.: 19/094,296

(22) Filed: Mar. 28, 2025

Related U.S. Application Data
(60) Provisional application No. 63/571,534, filed on Mar. 29, 2024.

(51) Int. Cl. A61K 36/81; A61K 31/198; A61K 31/4045; A61K 36/84

(52) U.S. Cl. CPC A61K 36/81; A61K 31/198; A61K 31/4045; A61K 36/84

(57) ABSTRACT

Sleep aid compositions are described herein. For example, this document provides sleep aid compositions containing melatonin, L-theanine, GABA (gamma-aminobutyric acid), and ashwagandha. Such compositions may be used to increase relaxation, feel relaxed more quickly, reduce stress, fall asleep, and/or stay asleep.

### CROSS-REFERENCE TO RELATED APPLICATIONS

This application claims priority from U.S. Provisional Application No. 63/571,534, filed on Mar. 29, 2024. The disclosure of the prior application is considered part of (and is incorporated by reference in) the disclosure of this application.

### TECHNICAL FIELD

This document relates to the field of sleep aid compositions. For example, this document relates to sleep aid compositions useful for one or more of relieving stress, enhancing mood, and supporting sleep.

### BACKGROUND

One-third of North American adults are not getting the sleep they need. The impact that lost sleep has on your health is significant. Sleep deprivation interferes with multiple systems in the body, including cognition, metabolism, immunity, and heart health. Accordingly, there is a continuing need to formulate compositions that support and/or improve sleep.

### SUMMARY

Provided herein are sleep aid compositions comprising melatonin; L-theanine; GABA (gamma-aminobutyric acid); and an ashwagandha extract. For example, provided herein are sleep aid compositions comprising melatonin; L-theanine present in an amount of about 150 mg to about 500 mg per serving; GABA (gamma-aminobutyric acid) present in an amount of about 150 mg to about 450 mg per serving; and an ashwagandha extract. In some embodiments, the melatonin is present in an amount of about 0.5 mg to about 15 mg per serving (e.g., per serving of the sleep aid composition). In some embodiments, the melatonin is present in an amount of about 1 mg to about 10 mg per serving. In some embodiments, the melatonin is present in an amount of about 5 mg per serving.

In some embodiments, the sleep aid composition is formulated for rapid release and sustained release of the melatonin. In some embodiments, about 20% to about 60% w/w of the melatonin is released within a first period of time. In some embodiments, about 40% w/w of the melatonin is released within a first period of time.

In some embodiments, the first period of time is about 30 minutes to about 1 hour. In some embodiments, the first period of time is about 1 hour.

In some embodiments, about 40% to about 80% w/w of the melatonin is released over a second period of time following the first period of time. In some embodiments, about 60% w/w of the melatonin is released within the second period of time.

In some embodiments, the first period of time is about 6 hours to about 8 hours. In some embodiments, the second period of time is about 7 hours.

In some embodiments, the L-theanine is present in an amount of about 100 mg to about 300 mg per serving (e.g., per serving of the sleep aid composition). In some embodiments, the L-theanine is present in an amount of about 200 mg per serving.

In some embodiments, the GABA is present in an amount of about 200 mg to about 400 mg per serving (e.g., per serving of the sleep aid composition). In some embodiments, the GABA is present in an amount of about 300 mg per serving.

In some embodiments, the ashwagandha extract is present in an amount of about 5 mg to about 600 mg per serving (e.g., per serving of the sleep aid composition). In some embodiments, the ashwagandha extract is present in an amount of about 100 mg to about 500 mg per serving. In some embodiments, the ashwagandha extract is present in an amount of about 128 mg per serving.

In some embodiments, the ashwagandha extract contains one or more withanolides. In some embodiments, the one or more withanolides are present in an amount of about 1% to about 12% w/w of the ashwagandha extract. In some embodiments, the one or more withanolides are present in an amount of about 5% w/w of the ashwagandha extract.

In some embodiments, the sleep aid composition further comprises a valerian root extract.

In some embodiments, the sleep aid composition is formulated as a tablet, a capsule, or a soft gel. In some embodiments, the sleep aid composition is formulated as a tablet. In some embodiments, the one tablet, capsule, or soft gel comprises a full serving of the composition.

Also provided herein are methods of reducing stress in a mammal, the method comprising administering to the mammal a composition of any of the compositions described herein.

Also provided herein are methods of improving sleep quality in a mammal, the method comprising administering to the mammal a composition of any of the compositions described herein.

Also provided herein are methods of decreasing sleep latency in a mammal, the method comprising administering to the mammal a composition of any of the compositions described herein.

Also provided herein are methods of increasing sleep duration, the method comprising administering to the mammal a composition of any of the compositions described herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the description below. Other features and advantages will be apparent from the description and from the claims.

### DETAILED DESCRIPTION

This document provides sleep aid compositions that contain melatonin, L-theanine, GABA (gamma-aminobutyric acid), and an ashwagandha extract. Such sleep aid compositions can be useful for one or more of increasing relaxation, feeling relaxed more quickly, reducing stress, falling asleep faster, and staying asleep longer (e.g., after consuming a sleep aid composition provided herein as compared to not taking a sleep aid composition provided herein).

### Melatonin

The sleep aid compositions provided herein include melatonin (also referred to as N-acetyl-5-methoxytryptamine). Melatonin is a hormone that can be produced by the pineal gland in the brain to help regulate sleep cycles. Any appropriate method can be used to obtain melatonin for use in the sleep aid compositions provided herein. For example, melatonin can be obtained by chemical synthesis.

The sleep aid compositions provided herein can contain any appropriate amount of melatonin. In some embodiments, melatonin is present in an amount of about 0.5 mg to about 15 mg per serving of a sleep aid composition provided herein. For example, melatonin can be present in an amount of about 0.5 mg to about 2 mg, about 0.5 mg to about 4 mg, about 0.5 mg to about 6 mg, about 0.5 mg to about 8 mg, about 0.5 mg to about 10 mg, about 0.5 mg to about 12 mg, about 0.5 mg to about 14 mg, about 2 mg to about 4 mg, about 2 mg to about 6 mg, about 2 mg to about 8 mg, about 2 mg to about 10 mg, about 2 mg to about 12 mg, about 2 mg to about 14 mg, about 4 mg to about 6 mg, about 4 mg to about 8 mg, about 4 mg to about 10 mg, about 4 mg to about 12 mg, about 4 mg to about 14 mg, about 6 mg to about 8 mg, about 6 mg to about 10 mg, about 6 mg to about 12 mg, about 6 mg to about 13 mg, about 6 mg to about 14 mg, about 6 mg to about 15 mg, about 8 mg to about 9 mg, about 8 mg to about 10 mg, about 8 mg to about 11 mg, about 8 mg to about 12 mg, about 8 mg to about 13 mg, about 8 mg to about 14 mg, about 8 mg to about 15 mg, about 10 mg to about 11 mg, about 10 mg to about 12 mg, about 10 mg to about 13 mg, about 10 mg to about 14 mg, about 10 mg to about 15 mg, about 12 mg to about 13 mg, about 12 mg to about 14 mg, or about 12 mg to about 15 mg per serving of a sleep aid composition provided herein. In some embodiments, melatonin is present in an amount of about 4 mg to about 6 mg per serving of a sleep aid composition provided herein. In some embodiments, melatonin is present in an amount of about 0.5 mg, about 1 mg, about 2 mg, about 3 mg, about 4 mg, about 5 mg, about 6, mg, about 7 mg, about 8 mg, about 9 mg, or about 10 mg per serving of a sleep aid composition provided herein. In some embodiments, melatonin is present in an amount of about 5 mg per serving of a sleep aid composition provided herein.

In some embodiments, melatonin is present in an amount of about 0.04% to about 1.25% w/w of a sleep aid composition provided herein. For example, melatonin can be present in an amount of about 0.04% to about 0.1%, about 0.04% to about 0.25%, about 0.04% to about 0.5%, about 0.04% to about 0.75%, about 0.04% to about 1%, about 0.1% to about 0.25%, about 0.1% to about 0.5%, about 0.1% to about 0.75%, about 0.1% to about 1%, about 0.1% to about 1.25%, about 0.25% to about 0.5%, about 0.25% to about 0.75%, about 0.25% to about 1%, about 0.25% to about 1.25%, about 0.5% to about 0.75%, about 0.5% to about 1%, about 0.5% to about 1.25%, about 0.75% to about 1%, about 0.75% to about 1.25%, or about 1% to about 1.25% w/w of a sleep aid composition provided herein. In some embodiments, melatonin is present in an amount of about 0.1% to about 0.8% w/w of a sleep aid composition provided herein. For example, melatonin is present in an amount of about 0.1%, about 0.2%, about 0.3%, about 0.4%, about 0.5%, about 0.6%, about 0.7%, or about 0.8% w/w of a sleep aid composition provided herein.

In some embodiments, a sleep aid composition provided herein can be formulated for rapid release of melatonin, sustained release of melatonin, or a combination thereof. For example, when a sleep aid composition provided herein is formulated for rapid release of melatonin, the melatonin can be released within a period of time (e.g., a first period of time) after taking a sleep aid composition provided herein. In some embodiments, when a sleep aid composition provided herein is formulated for sustained release of melatonin, the melatonin can be released gradually over a period of time (e.g., a second period of time) that is longer than the period of time for a rapid release formulation.

In some embodiments, the period of time for the release of melatonin in a rapid release formulation (e.g., the first period of time) is about 1 hour or less, e.g., about 1 hour, about 45 minutes, about 30 minutes, or about 15 minutes. In some embodiments, the period of time for the release of melatonin in a rapid release formulation (e.g., the first period of time) is about 1 hour. In some embodiments, the period of time for the release of melatonin in a sustained release formulation (e.g., the second period of time) is about 3 or more hours, e.g., about 3 hours, about 4 hours, about 5 hours, about 6 hours, about 7 hours, or about 8 hours. In some embodiments, the period of time for the release of melatonin in a sustained release formulation (e.g., the second period of time) is about 6 hours. In some embodiments, a second period of time begins when the first period of time ends.

In some embodiments, a sleep aid composition provided herein is formulated for a combination of rapid release and sustained release of melatonin. For example, a portion of the melatonin in a sleep aid composition provided herein can be released within a first period of time, and all or most of the remaining melatonin in the sleep aid composition can be released over a second period of time. In some embodiments, about 20% to about 60% w/w of the melatonin in a sleep aid composition provided herein is released within a first period of time. For example, about 20% to about 30%, about 20% to about 40%, about 20% to about 50%, about 30% to about 40%, about 30% to about 50%, about 30% to about 40%, or about 40% to about 50% w/w of the melatonin in a sleep aid composition provided herein can be released within a first period of time. In some embodiments, about 40% to about 80% w/w of the melatonin in a sleep aid composition provided herein is released over a second period of time. For example, about 40% to about 50%, about 40% to about 60%, about 40% to about 70%, about 50% to about 60%, about 50% to about 70%, about 50% to about 80%, about 60% to about 70%, about 60% to about 80%, or about 70% to about 80% w/w of the melatonin in a sleep aid composition provided herein can be released over a second period of time.

In some embodiments, about 30% to about 40% w/w of the melatonin in a sleep aid composition provided herein is released within a first period of time (e.g., about 1 hour) and about 50% to about 60% w/w of the melatonin in the sleep aid composition is released over a second period of time (e.g., about 50% to about 60% w/w of the melatonin is released over about 6 hours following the first period of time).

In some embodiments, about 30% to about 40% w/w of the melatonin in a sleep aid composition provided herein is released within a first period of time (e.g., about 1 hour) and about 50% to about 60% w/w of the melatonin in the sleep aid composition is released over a second period of time (e.g., about 50% to about 60% w/w of the melatonin is released over about 7 hours following the first period of time).

In some embodiments, about 40% w/w of the melatonin in a sleep aid composition provided herein is released within a first period of time (e.g., about 1 hour) and about 60% w/w of the melatonin in the sleep aid composition is released over a second period of time (e.g., about 60% w/w of the melatonin is released over about 6 hours following the first period of time).

In some embodiments, about 40% w/w of the melatonin in a sleep aid composition provided herein is released within a first period of time (e.g., about 1 hour) and about 60% w/w of the melatonin in the sleep aid composition is released over a second period of time (e.g., about 60% w/w of the melatonin is released over about 7 hours following the first period of time).

In some embodiments, where a sleep aid composition is formulated for rapid release of melatonin, the melatonin is present in an amount of about 0.1 mg to about 10 mg per serving of a sleep aid composition provided herein. For example, the melatonin can be present in an amount of about 0.1 mg to about 1 mg, about 0.1 mg to about 2 mg, about 0.1 mg to about 3 mg, about 0.1 mg to about 4 mg, about 0.1 mg to about 5 mg, about 0.1 mg to about 6 mg, about 0.1 mg to about 7 mg, about 0.1 mg to about 8 mg, about 0.1 mg to about 9 mg, about 1 mg to about 2 mg, about 1 mg to about 3 mg, about 1 mg to about 4 mg, about 1 mg to about 5 mg, about 1 mg to about 6 mg, about 1 mg to about 7 mg, about 1 mg to about 8 mg, about 1 mg to about 9 mg, about 2 mg to about 3 mg, about 2 mg to about 4 mg, about 2 mg to about 5 mg, about 2 mg to about 6 mg, about 2 mg to about 7 mg, about 2 mg to about 8 mg, about 2 mg to about 9 mg, about 2 mg to about 10 mg, about 3 mg to about 4 mg, about 3 mg to about 5 mg, about 3 mg to about 6 mg, about 3 mg to about 7 mg, about 3 mg to about 8 mg, about 3 mg to about 9 mg, about 3 mg to about 10 mg, about 4 mg to about 5 mg, about 4 mg to about 6 mg, about 4 mg to about 7 mg, about 4 mg to about 8 mg, about 4 mg to about 9 mg, about 4 mg to about 10 mg, about 5 mg to about 6 mg, about 5 mg to about 7 mg, about 5 mg to about 8 mg, about 5 mg to about 9 mg, about 5 mg to about 10 mg, about 6 mg to about 7 mg, about 6 mg to about 8 mg, about 6 mg to about 9 mg, about 6 mg to about 10 mg, about 7 mg to about 8 mg, about 7 mg to about 9 mg, about 7 mg to about 10 mg, about 8 mg to about 9 mg, about 8 mg to about 10 mg, or about 9 mg to about 10 mg per serving of a sleep aid composition provided herein. In some embodiments, the melatonin is present in an amount of about 0.1 mg to about 5 mg per serving of a sleep aid composition provided herein. In some embodiments, the melatonin is present in an amount of about 0.1 mg, about 1 mg, about 2 mg, about 3 mg, about 4 mg, about 5 mg, about 6 mg, about 7 mg, about 8 mg, about 9 mg, or about 10 mg per serving of a sleep aid composition provided herein.

In some embodiments, where a sleep aid composition is formulated for rapid release of melatonin, the melatonin is present in an amount of about 0.008% to about 0.8% w/w of a sleep aid composition provided herein. For example, the melatonin can be present in an amount of about 0.008% to about 0.01%, 0.008% to about 0.05%, about 0.008% to about 0.1%, about 0.008% to about 0.2%, about 0.008% to about 0.3%, about 0.008% to about 0.4%, about 0.008% to about 0.5%, about 0.008% to about 0.6%, about 0.008% to about 0.7%, about 0.01% to about 0.05%, about 0.01% to about 0.1%, about 0.01% to about 0.2%, about 0.01% to about 0.3%, about 0.01% to about 0.4%, about 0.01% to about 0.5%, about 0.01% to about 0.6%, about 0.01% to about 0.7%, about 0.01% to about 0.8%, about 0.05% to about 0.1%, about 0.05% to about 0.2%, about 0.05% to about 0.3%, about 0.05% to about 0.4%, about 0.05% to about 0.5%, about 0.05% to about 0.6%, about 0.05% to about 0.7%, about 0.05% to about 0.8%, about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.3% to about 0.4%, about 0.3% to about 0.5%, about 0.3% to about 0.6%, about 0.3% to about 0.7%, about 0.3% to about 0.8%, about 0.4% to about 0.5%, about 0.4% to about 0.6%, about 0.4% to about 0.7%, about 0.4% to about 0.8%, about 0.5% to about 0.6%, about 0.5% to about 0.7%, about 0.5% to about 0.8%, about 0.6% to about 0.7%, about 0.6% to about 0.8%, or about 0.7% to about 0.8% w/w of a sleep aid composition provided herein. In some embodiments, the melatonin is present in an amount of about 0.008%, about 0.1%, about 0.2%, about 0.3%, about 0.4%, about 0.5%, about 0.6%, about 0.7%, or about 0.8% w/w of a sleep aid composition provided herein.

In some embodiments, where a sleep aid composition is formulated for sustained release of melatonin, the melatonin is present in an amount of about 0.5 mg to about 5 mg per serving of a sleep aid composition provided herein. For example, the melatonin can be present in an amount of about 0.5 mg to about 1 mg, about 0.5 mg to about 2 mg, about 0.5 mg to about 3 mg, about 0.5 mg to about 4 mg, about 1 mg to about 2 mg, about 1 mg to about 3 mg, about 1 mg to about 4 mg, about 1 mg to about 5 mg, about 2 mg to about 3 mg, about 2 mg to about 4 mg, about 2 mg to about 5 mg, about 3 mg to about 4 mg, about 3 mg to about 5 mg, or about 4 mg to about 5 mg per serving of a sleep aid composition provided herein. In some embodiments, the melatonin is present in an amount of about 0.5 mg, about 1 mg, about 2 mg, about 3 mg, about 4 mg, or about 5 mg per serving of a sleep aid composition provided herein.

In some embodiments, where a sleep aid composition is formulated for sustained release of melatonin, the melatonin is present in an amount of about 0.04% to about 0.4% w/w of a sleep aid composition provided herein. For example, the melatonin can be present in an amount of 0.04% to about 0.05%, about 0.04% to about 0.1%, about 0.04% to about 0.2%, about 0.04% to about 0.3%, about 0.04% to about 0.05%, about 0.04% to about 0.1%, about 0.04% to about 0.2%, about 0.04% to about 0.3%, about 0.04% to about 0.4%, about 0.05% to about 0.1%, about 0.05% to about 0.2%, about 0.05% to about 0.3%, about 0.05% to about 0.4%, about 0.1% to about 0.2%, about 0.1% to about 0.3%, about 0.1% to about 0.4%, about 0.2% to about 0.3%, about 0.2% to about 0.4%, or about 0.3% to about 0.4% w/w of a sleep aid composition provided herein. In some embodiments, the melatonin is present in an amount of about 0.04%, about 0.1%, about 0.2%, about 0.3%, or about 0.4% w/w of a sleep aid composition provided herein.

### L-Theanine

The sleep aid compositions provided herein include L-theanine. L-theanine is an amino acid commonly found in tea leaves. L-theanine can help reduce anxiety and/or improve sleep quality. Any appropriate method can be used to obtain L-theanine for use in the sleep aid compositions provided herein. For example, L-theanine can be obtained by chemical synthesis or extracted from tea (e.g., green tea leaves).

The sleep aid compositions provided herein can contain any appropriate amount of L-theanine. In some embodiments, L-theanine is present in an amount of about 50 mg to about 500 mg per serving of a sleep aid composition provided herein. For example, L-theanine can be present in an amount of about 50 mg to about 100 mg, about 50 mg to about 200 mg, about 50 mg to about 300 mg, about 50 mg to about 400 mg, about 100 mg to about 200 mg, about 100 mg to about 300 mg, about 100 mg to about 400 mg, about 100 mg to about 500 mg, about 200 mg to about 300 mg, about 200 mg to about 400 mg, about 200 mg to about 500 mg, about 300 mg to about 400 mg, about 300 mg to about 500 mg, or about 400 mg to about 500 mg per serving of a sleep aid composition provided herein. In some embodiments, L-theanine is present in an amount of about 100 mg to about 300 mg per serving of a sleep aid composition provided herein.

In some embodiments, L-theanine is present in an amount of about 150 mg to about 500 mg per serving of a sleep aid composition provided herein. In some embodiments, L-theanine is present in an amount of about 150 mg to about 300 mg per serving of a sleep aid composition provided herein. In some embodiments, L-theanine is present in an amount of about 150 mg to about 250 mg per serving of a sleep aid composition provided herein. In some embodiments, L-theanine is present in an amount of about 175 mg to about 225 mg per serving of a sleep aid composition provided herein.

In some embodiments, L-theanine is present in an amount of about 50 mg, about 100 mg, about 200 mg, about 300 mg, about 400 mg, or about 500 mg per serving of a sleep aid composition provided herein. In some embodiments, L-theanine is present in an amount of about 200 mg per serving of a sleep aid composition provided herein.

In some embodiments, L-theanine is present in an amount of about 4% to about 40% w/w of a sleep aid composition provided herein. For example, L-theanine can be present in an amount of about 4% to about 10%, about 4% to about 15%, about 4% to about 20%, about 4% to about 25%, about 4% to about 30%, about 4% to about 35%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% w/w of a sleep aid composition provided herein. In some embodiments, L-theanine is present in an amount of about 8% to about 25% w/w of a sleep aid composition provided herein. For example, L-theanine can be present in an amount of about 10%, about 15%, about 16%, about 17%, about 18%, about 19%, about 20%, or about 25% w/w of a sleep aid composition provided herein.

### GABA

The sleep aid compositions provided herein include GABA (gamma-aminobutyric acid or γ-aminobutyric acid). GABA is a neurotransmitter that can, for example, promote relaxation, have a calming effect on the nervous system, help regulate sleep cycles, and/or improve sleep. Any appropriate method can be used to obtain GABA for use in the sleep aid compositions provided herein. For example, GABA can be obtained by chemical synthesis.

The sleep aid compositions provided herein can contain any appropriate amount of GABA. In some embodiments, GABA is present in an amount of about 1 mg to about 3000 mg per serving of a sleep aid composition provided herein. For example, GABA can be present in an amount of about 1 mg to about 400 mg, about 1 mg to about 800 mg, about 1 mg to about 1200 mg, about 1 mg to about 1600 mg, about 1 mg to about 2000 mg, about 1 mg to about 2400 mg, about 1 mg to about 2800 mg, about 400 mg to about 800 mg, about 400 mg to about 1200 mg, about 400 mg to about 1600 mg, about 400 mg to about 2000 mg, about 400 mg to about 2400 mg, about 400 mg to about 2800 mg, about 400 mg to about 3000 mg, about 800 mg to about 1200 mg, about 800 mg to about 1600 mg, about 800 mg to about 2000 mg, about 800 mg to about 2400 mg, about 800 mg to about 2800 mg, about 800 mg to about 3000 mg, about 1200 mg to about 1600 mg, about 1200 mg to about 2000 mg, about 1200 mg to about 2400 mg, about 1200 mg to about 2800 mg, about 1200 mg to about 3000 mg, about 1600 mg to about 2000 mg, about 1600 mg to about 2400 mg, about 1600 mg to about 2800 mg, about 1600 mg to about 3000 mg, about 2000 mg to about 2400 mg, about 2000 mg to about 2800 mg, about 2000 mg to about 3000 mg, about 2400 mg to about 2800 mg, about 2400 mg to about 3000 mg, or about 2800 mg to about 3000 mg per serving of a sleep aid composition provided herein. In some embodiments, GABA is present in an amount of about 100 mg to about 500 mg per serving of a sleep aid composition provided herein.

In some embodiments, GABA is present in an amount of about 150 mg to about 450 mg per serving of a sleep aid composition provided herein. In some embodiments, GABA is present in an amount of about 200 mg to about 400 mg per serving of a sleep aid composition provided herein. In some embodiments, GABA is present in an amount of about 250 mg to about 350 mg per serving of a sleep aid composition provided herein. In some embodiments, GABA is present in an amount of about 275 mg to about 325 mg per serving of a sleep aid composition provided herein.

In some embodiments, GABA is present in an amount of about 1 mg, about 200 mg, about 250 mg, about 300 mg, about 350 mg, about 400 mg, about 600 mg, about 800 mg, about 1000 mg, about 1200 mg, about 1400 mg, about 1600 mg, about 1800 mg, about 2000 mg, about 2200 mg, about 2400 mg, about 2600 mg, about 2800 mg, or about 3000 mg per serving of a sleep aid composition provided herein. In some embodiments, GABA is present in an amount of about 300 mg per serving of a sleep aid composition provided herein.

In some embodiments, GABA is present in an amount of about 8% to about 40% w/w of a sleep aid composition provided herein. For example, GABA can be present in an amount of 8% to about 10%, about 8% to about 15%, about 8% to about 20%, about 8% to about 25%, about 8% to about 30%, about 8% to about 35%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40% w/w of a sleep aid composition provided herein. In some embodiments, GABA is present in an amount of about 10%, 15%, 20%, 25%, 30%, 35% or 40% w/w of a sleep aid composition provided herein. In some embodiments, GABA is present in an amount of about 25% w/w of a sleep aid composition provided herein.

### Ashwagandha

The sleep aid compositions provided herein include ashwagandha. Ashwagandha (Withania somnifera) is an evergreen shrub found in tropical and subtropical areas of Asia, Africa, and Europe. It has been traditionally used for reducing stress.

In some embodiments, the sleep aid compositions provided herein can include ashwagandha whole root powder and/or an ashwagandha extract. An ashwagandha extract can be in the form of a liquid (e.g., an aqueous solution), a solid (e.g., a particulate or a powder), or a semi-solid form (e.g., a gel or an emulsion). Any appropriate method can be used to obtain ashwagandha for use in the sleep aid compositions provided herein. For example, ashwagandha whole root powder can be prepared by drying and grinding the root of the ashwagandha plant. As another example, an alcohol (e.g., ethanol) solution can be used to obtain an ashwagandha extract from ashwagandha roots and/or ashwagandha leaves.

In some embodiments, a sleep aid composition as provided herein includes ashwagandha whole root powder. The sleep aid compositions provided herein can contain any appropriate amount of ashwagandha whole root powder.

In some embodiments, ashwagandha whole root powder is present in an amount of about 2 g to about 6 g per serving of a sleep aid composition provided herein. For example, ashwagandha whole root powder can be present in an amount of about 2 g to about 3 g, about 2 g to about 4 g, about 2 g to about 5 g, about 3 g to about 4 g, about 3 g to about 5 g, about 3 g to about 6 g, about 4 g to about 5 g, about 4 g to about 6 g, or about 5 g to about 6 g per serving of a sleep aid composition provided herein.

In some embodiments, a sleep aid composition as provided herein includes an ashwagandha extract. In some embodiments, an ashwagandha extract is an extract from ashwagandha leaves and/or ashwagandha roots. In some embodiments, an ashwagandha extract is an ashwagandha root extract. For example, an ashwagandha root extract can be an extract where no ashwagandha leaves were used in producing the extract. In some embodiments, the proportions of compounds in an ashwagandha extract are similar to their proportions found naturally (e.g., in ashwagandha leaves and/or ashwagandha roots).

The sleep aid compositions provided herein can contain any appropriate amount of an ashwagandha extract. In some embodiments, an ashwagandha extract is present in an amount of about 5 mg to about 600 mg per serving of a sleep aid composition provided herein. For example, about 5 mg to about 50 mg, about 5 mg to about 100 mg, about 5 mg to about 150 mg, about 5 mg to about 200 mg, about 5 mg to about 250 mg, about 5 mg to about 300 mg, about 5 mg to about 350 mg, about 5 mg to about 400 mg, about 5 mg to about 450 mg, about 5 mg to about 500 mg, about 5 mg to about 550 mg, about 50 mg to about 100 mg, about 50 mg to about 150 mg, about 50 mg to about 200 mg, about 50 mg to about 250 mg, about 50 mg to about 300 mg, about 50 mg to about 350 mg, about 50 mg to about 400 mg, about 50 mg to about 450 mg, about 50 mg to about 500 mg, about 50 mg to about 550 mg, about 50 mg to about 600 mg, about 100 mg to about 150 mg, about 100 mg to about 200 mg, about 100 mg to about 250 mg, about 100 mg to about 300 mg, about 100 mg to about 350 mg, about 100 mg to about 400 mg, about 100 mg to about 450 mg, about 100 mg to about 500 mg, about 100 mg to about 550 mg, about 100 mg to about 600 mg, about 150 mg to about 200 mg, about 150 mg to about 250 mg, about 150 mg to about 300 mg, about 150 mg to about 350 mg, about 150 mg to about 400 mg, about 150 mg to about 450 mg, about 150 mg to about 500 mg, about 150 mg to about 550 mg, about 150 mg to about 600 mg, about 200 mg to about 250 mg, about 200 mg to about 300 mg, about 200 mg to about 350 mg, about 200 mg to about 400 mg, about 200 mg to about 450 mg, about 200 mg to about 500 mg, about 200 mg to about 550 mg, about 200 mg to about 600 mg, about 250 mg to about 300 mg, about 250 mg to about 350 mg, about 250 mg to about 400 mg, about 250 mg to about 450 mg, about 250 mg to about 500 mg, about 250 mg to about 550 mg, about 250 mg to about 600 mg, about 300 mg to about 350 mg, about 300 mg to about 400 mg, about 300 mg to about 450 mg, about 300 mg to about 500 mg, about 300 mg to about 550 mg, about 300 mg to about 600 mg, about 350 mg to about 400 mg, about 350 mg to about 450 mg, about 350 mg to about 500 mg, about 350 mg to about 550 mg, about 350 mg to about 600 mg, about 400 mg to about 450 mg, about 400 mg to about 500 mg, about 400 mg to about 550 mg, about 400 mg to about 600 mg, about 450 mg to about 500 mg, about 450 mg to about 550 mg, about 450 mg to about 600 mg, about 500 mg to about 550 mg, about 500 mg to about 600 mg, or about 550 mg to about 600 mg per serving of a sleep aid composition provided herein. In some embodiments, an ashwagandha extract is present in an amount of about 100 mg to about 500 mg per serving of a sleep aid composition provided herein. In some embodiments, an ashwagandha extract is present in an amount of about 100 mg, about 125 mg, about 130 mg, about 135 mg, about 140 mg, about 150 mg, about 175 mg, about 200 mg, about 250 mg, about 300 mg, about 350 mg, about 400 mg, about 450 mg, or about 500 mg per serving of a sleep aid composition provided herein. In some embodiments, an ashwagandha extract is present in an amount of about 128 mg per serving of a sleep aid composition provided herein.

In some embodiments, an ashwagandha extract is present in an amount of about 0.4% to about 50% w/w of a sleep aid composition provided herein. For example, an ashwagandha extract can be present in an amount of about 0.4% to about 1%, about 0.4% to about 5%, about 0.4% to about 10%, about 0.4% to about 15%, about 0.4% to about 20%, about 0.4% to about 25%, about 0.4% to about 30%, about 0.4% to about 35%, about 0.4% to about 40%, about 0.4% to about 45%, about 1% to about 5%, about 1% to about 10%, about 1% to about 15%, about 1% to about 20%, about 1% to about 25%, about 1% to about 30%, about 1% to about 35%, about 1% to about 40%, about 1% to about 45%, about 1% to about 50%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, or about 35% to about 40% w/w of a sleep aid composition provided herein. In some embodiments, an ashwagandha extract is present in an amount of about 8% to 40% w/w of a sleep aid composition provided herein. In some embodiments, an ashwagandha extract is present in an amount of about 8% to 12% w/w of a sleep aid composition provided herein. In some embodiments, an ashwagandha extract is present in an amount of about 5%, about 10%, about 15%, about 20%, about 25%, about 30%, about 35%, or about 40% w/w of a sleep aid composition provided herein. In some embodiments, an ashwagandha extract is present in an amount of about 11% w/w of a sleep aid composition provided herein.

Ashwagandha whole root powders and ashwagandha extracts can contain one or more withanolides. Withanolides include compounds consisting of a steroid backbone bound to a lactone or a lactone derivative. Non-limiting examples of a withanolide include withaferin A, salpichrolid A, nicandrenone-1, ixocarpalacton A, withaperuvin B, withangulatin C, acnistin I, physalin X, jaborosalactol 18, physalin C, jaborosalactone 7, salpichrolide G, jaborosalactone 37, jaborosalactone 43, subtrifloralactone E, tubonolide A, withametelinone, withaphysalin N, isocarpalactone B, perulactone C, jaborosalactone 15, subtrifloralactone K, taccalonolide A, jaborosalactol 24, physanolide A, exodeconolide A, withanoside VII, 27-hydroxywithanone, dihydrowithaferin A, viscosalactone B, withanoside IV, withanoside V, withaferin A, 12-deoxywithastramonolide, withanolide A, withanone, and withanolide B. In some embodiments, the one or more withanolides include withaferin A.

In some embodiments, ashwagandha whole root powder contains one or more withanolides present in an amount of at least about 0.4%, e.g., at least about 0.5%, at least about 0.6%, at least about 0.7%, at least about 0.8%, at least about 0.9%, at least about 1%, or at least about 1.2%, w/w of the ashwagandha whole root powder. In some embodiments, ashwagandha whole root powder contains one or more withanolides present in an amount of about 0.5% to about 1.5% w/w of the ashwagandha whole root powder. For example, ashwagandha whole root powder can contain one or more withanolides present in an amount of about 0.5% to about 0.8%, about 0.5% to about 1%, about 0.5% to about 1.2%, about 0.8% to about 1%, about 0.8% to about 1.2%, about 0.8% to about 1.5%, about 1% to about 1.2%, about 1% to about 1.5%, or about 1.2% to about 1.5% w/w of the ashwagandha whole root powder.

In some embodiments, an ashwagandha extract contains one or more withanolides present in an amount of at least about 4%, e.g., at least about 5%, at least about 6%, at least about 7%, at least about 8%, at least about 9%, or at least about 10% w/w of the ashwagandha extract. In some embodiments, the ashwagandha extract contains one or more withanolides present in an amount of about 1% to about 12% w/w of the ashwagandha extract. For example, the ashwagandha extract can contain one or more withanolides present in an amount of about 1% to about 2%, about 1% to about 3%, about 1% to about 4%, about 1% to about 5%, about 1% to about 6%, about 1% to about 7%, about 1% to about 8%, about 1% to about 9%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10% w/w of the ashwagandha extract. In some embodiments, the ashwagandha extract contains one or more withanolides present in an amount of about 5% w/w of the ashwagandha extract.

In some embodiments, the one or more withanolides are present in an amount of about 0.25 mg to about 30 mg per serving of a sleep aid composition provided herein. For example, the one or more withanolides are present in an amount of about 0.25 mg to about 1 mg, about 0.25 mg to about 5 mg, about 0.25 mg to about 10 mg, about 0.25 mg to about 15 mg, about 0.25 mg to about 20 mg, about 0.25 mg to about 25 mg, about 1 mg to about 5 mg, about 1 mg to about 10 mg, about 1 mg to about 15 mg, about 1 mg to about 20 mg, about 1 mg to about 25 mg, about 1 mg to about 30 mg, about 5 mg to about 10 mg, about 5 mg to about 15 mg, about 5 mg to about 20 mg, about 5 mg to about 25 mg, about 5 mg to about 30 mg, about 10 mg to about 15 mg, about 10 mg to about 20 mg, about 10 mg to about 25 mg, about 10 mg to about 30 mg, about 15 mg to about 20 mg, about 15 mg to about 25 mg, about 15 mg to about 30 mg, about 20 mg to about 25 mg, about 20 mg to about 30 mg, or about 25 mg to about 30 mg per serving of a sleep aid composition provided herein. In some embodiments, the one or more withanolides are present in an amount of about 5 mg to about 25 mg per serving of a sleep aid composition provided herein. In some embodiments, the one or more withanolides are present in an amount of about 5 mg, about 10 mg, about 15 mg, about 20 mg, or about 25 mg per serving of a sleep aid composition provided herein. In some embodiments, the one or more withanolides are present in an amount of about 6.25 mg per serving of a sleep aid composition provided herein.

In some embodiments, the one or more withanolides are present in an amount of about 0.04% to about 2% w/w of a sleep aid composition provided herein. For example, the one or more withanolides can be present in an amount of about 0.04% to about 0.08%, about 0.04% to about 0.2%, about 0.04% to about 0.4%, about 0.04% to about 0.6%, about 0.04% to about 0.8%, about 0.04% to about 1%, about 0.04% to about 1.2%, about 0.04% to about 1.4%, about 0.04% to about 1.6%, about 0.04% to about 1.8%, about 0.08% to about 0.2%, about 0.08% to about 0.4%, about 0.08% to about 0.6%, about 0.08% to about 0.8%, about 0.08% to about 1%, about 0.08% to about 1.2%, about 0.08% to about 1.4%, about 0.08% to about 1.6%, about 0.08% to about 1.8%, about 0.08% to about 2%, about 0.2% to about 0.4%, about 0.2% to about 0.6%, about 0.2% to about 0.8%, about 0.2% to about 1%, about 0.2% to about 1.2%, about 0.2% to about 1.4%, about 0.2% to about 1.6%, about 0.2% to about 1.8%, about 0.2% to about 2%, about 0.4% to about 0.6%, about 0.4% to about 0.8%, about 0.4% to about 1%, about 0.4% to about 1.2%, about 0.4% to about 1.4%, about 0.4% to about 1.6%, about 0.4% to about 1.8%, about 0.4% to about 2%, about 0.6% to about 0.8%, about 0.6% to about 1%, about 0.6% to about 1.2%, about 0.6% to about 1.4%, about 0.6% to about 1.6%, about 0.6% to about 1.8%, about 0.6% to about 2%, about 0.8% to about 1%, about 0.8% to about 1.2%, about 0.8% to about 1.4%, about 0.8% to about 1.6%, about 0.8% to about 1.8%, about 0.8% to about 2%, about 1% to about 1.2%, about 1% to about 1.4%, about 1% to about 1.6%, about 1% to about 1.8%, about 1% to about 2%, about 1.2% to about 1.4%, about 1.2% to about 1.6%, about 1.2% to about 1.8%, about 1.2% to about 2%, about 1.4% to about 1.6%, about 1.4% to about 1.8%, about 1.4% to about 2%, about 1.6% to about 1.8%, about 1.6% to about 2%, or about 1.8% to about 2% w/w of a sleep aid composition provided herein. In some embodiments, the one or more withanolides are present in an amount of about 0.1%, about 0.2%, about 0.3%, about 0.4%, about 0.5%, about 0.6%, about 0.7%, about 0.8%, or about 1% w/w of a sleep aid composition provided herein. In some embodiments, the one or more withanolides are present in an amount of about 0.5% w/w of a sleep aid composition provided herein.

### Valerian Root Extract

The sleep aid compositions provided herein can further include valerian root extract. Valerian (Valeriana officinalis) is a perennial plant native to Europe and Asia and naturalized in North America. Valerian root extract may help improve sleep.

A valerian root extract can be in the form of a liquid (e.g., an aqueous solution), a solid (e.g., a particulate or a powder), or a semi-solid form (e.g., a gel or an emulsion). Any appropriate method can be used to obtain a valerian root extract for use in the sleep aid compositions provided herein. For example, an aqueous solution can be used to obtain a valerian root extract from valerian.

The sleep aid compositions provided herein can contain any appropriate amount of a valerian root extract. In some embodiments, a valerian root extract is present in an amount of about 100 mg to about 800 mg per serving of a sleep aid composition provided herein. For example, a valerian root extract can be present in an amount of about 100 mg to about 200 mg, about 100 mg to about 300 mg, about 100 mg to about 400 mg, about 100 mg to about 500 mg, about 100 mg to about 600 mg, about 100 mg to about 700 mg, about 200 mg to about 300 mg, about 200 mg to about 400 mg, about 200 mg to about 500 mg, about 200 mg to about 600 mg, about 200 mg to about 700 mg, about 200 mg to about 800, about 300 mg to about 400 mg, about 300 mg to about 500 mg, about 300 mg to about 600 mg, about 300 mg to about 700 mg, about 300 mg to about 800 mg, about 400 mg to about 500 mg, about 400 mg to about 600 mg, about 400 mg to about 700 mg, about 400 mg to about 800 mg, about 500 mg to about 600 mg, about 500 mg to about 700 mg, about 500 mg to about 800 mg, about 600 mg to about 700 mg, about 600 mg to about 800 mg, or about 700 mg to about 800 mg per serving of a sleep aid composition provided herein. In some embodiments, a valerian root extract is present in an amount of about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg per serving of a sleep aid composition provided herein.

In some embodiments, a sleep aid composition provided herein contains an amount of valerian root extract equivalent to about 1 g to about 9 g of valerian root material per serving (e.g., a sleep aid composition provided herein can contain an amount of valerian root extract equivalent to about 1 g to about 3 g, 1 g to about 6 g, about 3 g to about 6 g, about 6 g to about 9 g of valerian root powder per serving).

In some embodiments, a valerian root extract can have an extraction ratio of about 9:1, meaning that each milligram of extract is equivalent to 9 milligrams of the plant in its natural state.

Valerian root extracts can contain valerenic acid.

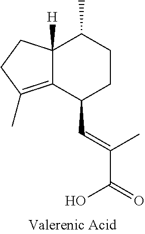 [SMILES: CC1=C2[C@H](/C=C(\C)C(=O)O)CC[C@@H](C)[C@H]2CC1]

In some embodiments, a valerian root extract contains valerenic acid present in an amount of about 0.1% to about 0.8% w/w of the valerian root extract. For example, the valerian root extract can contain valerenic acid present in an amount of about 0.1% to about 0.15%, about 0.1% to about 0.2%, about 0.1% to about 0.25%, about 0.1% to about 0.3%, about 0.1% to about 0.35%, about 0.1% to about 0.4%, about 0.1% to about 0.45%, about 0.1% to about 0.5%, about 0.1% to about 0.55%, about 0.1% to about 0.6%, about 0.1% to about 0.65%, about 0.1% to about 0.7%, about 0.1% to about 0.75%, about 0.15% to about 0.2%, about 0.15% to about 0.25%, about 0.15% to about 0.3%, about 0.15% to about 0.35%, about 0.15% to about 0.4%, about 0.15% to about 0.45%, about 0.15% to about 0.5%, about 0.15% to about 0.55%, about 0.15% to about 0.6%, about 0.15% to about 0.65%, about 0.15% to about 0.7%, about 0.15% to about 0.75%, about 0.15% to about 0.80%, about 0.2% to about 0.25%, about 0.2% to about 0.3%, about 0.2% to about 0.35%, about 0.2% to about 0.4%, about 0.2% to about 0.45%, about 0.2% to about 0.5%, about 0.2% to about 0.55%, about 0.2% to about 0.6%, about 0.2% to about 0.65%, about 0.2% to about 0.7%, about 0.2% to about 0.75%, about 0.2% to about 0.80%, about 0.25% to about 0.3%, about 0.25% to about 0.35%, about 0.25% to about 0.4%, about 0.25% to about 0.45%, about 0.25% to about 0.5%, about 0.25% to about 0.55%, about 0.25% to about 0.6%, about 0.25% to about 0.65%, about 0.25% to about 0.7%, about 0.25% to about 0.75%, about 0.25% to about 0.80%, about 0.3% to about 0.35%, about 0.3% to about 0.4%, about 0.3% to about 0.45%, about 0.3% to about 0.5%, about 0.3% to about 0.55%, about 0.3% to about 0.6%, about 0.3% to about 0.65%, about 0.3% to about 0.7%, about 0.3% to about 0.75%, about 0.3% to about 0.80%, about 0.35% to about 0.4%, about 0.35% to about 0.45%, about 0.35% to about 0.5%, about 0.35% to about 0.55%, about 0.35% to about 0.6%, about 0.35% to about 0.65%, about 0.35% to about 0.7%, about 0.35% to about 0.75%, about 0.35% to about 0.80%, about 0.4% to about 0.45%, about 0.4% to about 0.5%, about 0.4% to about 0.55%, about 0.4% to about 0.6%, about 0.4% to about 0.65%, about 0.4% to about 0.7%, about 0.4% to about 0.75%, about 0.4% to about 0.80%, about 0.45% to about 0.5%, about 0.45% to about 0.55%, about 0.45% to about 0.6%, about 0.45% to about 0.65%, about 0.45% to about 0.7%, about 0.45% to about 0.75%, about 0.45% to about 0.80%, about 0.5% to about 0.55%, about 0.5% to about 0.6%, about 0.5% to about 0.65%, about 0.5% to about 0.7%, about 0.5% to about 0.75%, about 0.5% to about 0.80%, about 0.55% to about 0.6%, about 0.55% to about 0.65%, about 0.55% to about 0.7%, about 0.55% to about 0.75%, about 0.55% to about 0.80%, about 0.6% to about 0.65%, about 0.6% to about 0.7%, about 0.6% to about 0.75%, about 0.6% to about 0.80%, about 0.65% to about 0.7%, about 0.65% to about 0.75%, about 0.65% to about 0.80%, about 0.7% to about 0.75%, or about 0.7% to about 0.80% w/w of the valerian root extract. In some embodiments, the valerian root extract contains valerenic acid present in an amount of about 0.1%, 0.2%, 0.3%, 0.4%, 0.5%, 0.6%, 0.7%, or 0.8% w/w of the valerian root extract.

Combinations of the melatonin, L-theanine, GABA, and ashwagandha of a sleep aid composition provided herein may have not only a beneficial effect but may also have a synergistic effect. For example, two or more of the melatonin, L-theanine, GABA, and ashwagandha may have an effect that is greater when used together than the sum of the effects that results from using the two or more components separately. See, e.g., Kim et al. Pharm Biol. 57(1): 65-73 (2019).

In some embodiments, the combination of GABA and L-theanine in a sleep aid composition provided herein has a synergistic effect. For example, the combination of GABA and L-theanine in a sleep aid composition provided herein can improve sleep quality and/or sleep duration more than when either GABA or L-theanine is used separately. In some embodiments, GABA and L-theanine are present in a sleep aid composition provided herein at a ratio of about 5:1 to about 1:3, wherein the ratio is the amount of GABA to the amount of L-theanine (e.g., in mg per serving) in a sleep aid composition provided herein. In some embodiments, GABA and L-theanine are present in a sleep aid composition provided herein at a ratio of about 3:2.

In some embodiments, a sleep aid composition provided herein is a composition that includes the ingredients listed in Table 1.

TABLE 1

| | Amount |
|---|---|
| Ingredient | (mg per serving) |
| GABA | 100 to 500 |
| L-Theanine | 100 to 300 |
| Rapid-release melatonin | 1 to 5 |
| Sustained-release melatonin | 0.5 to 5 |
| Ashwagandha extract | 100 to 500 |

In some embodiments, the ashwangdha extract contains at least about 5% withanolides. In some embodiments, the ashwangdha extract contains about 5% withanolides.

In some embodiments, a sleep aid composition provided herein is a composition that includes the ingredients listed in Table 2.

TABLE 2

| | Amount |
|---|---|
| Ingredient | (mg per serving) |
| GABA | 300 |
| L-Theanine | 200 |
| Melatonin | 5 |
| Ashwagandha extract | 127.5 |

In some embodiments, the sleep aid composition is formulated for both rapid and sustained release of the melatonin. In some embodiments, about 40% w/w of the melatonin is released within a first period of time (e.g., about 1 hour) and about 60% w/w of the melatonin is released over a second period of time (e.g., about 60% w/w of the melatonin is released over about 7 hours following the first period of time).

In some embodiments, the ashwagandha extract contains at least about 5% withanolides. In some embodiments, the ashwagandha extract contains about 5% withanolides. In some embodiments, the sleep aid composition contains about 6.25 mg withanolides.

A sleep aid composition provided herein can be formulated for oral use and can further include one or more suitable excipients, sweetening agents, flavorants, colorants, preservatives, and/or other ingredients. Non-limiting examples of oral formulations include tablets, troches, lozenges, aqueous or oil suspensions, non-aqueous solutions, dispersible powders or granules (including micronized particles or nanoparticles), emulsions, hard or soft capsules, syrups, and elixirs. Compositions intended for oral use can be prepared according to any appropriate method, including standard methods for the manufacture of pharmaceutical compositions. Preparations for oral use can be suitably formulated to give controlled release of one or more compounds.

Liquid preparations for oral administration can take the form of, for example, solutions, syrups, or suspension, or they can be presented as a dry product for reconstitution with saline or other suitable liquid vehicle before use. In some embodiments, liquid preparations can contain pharmaceutically acceptable additives such as suspending agents, emulsifying agents, non-aqueous vehicles, preservatives, buffer salts, flavoring agents, coloring agents, and sweetening agents as appropriate.

Tablets, capsules, or soft gels can be prepared with one or more suitable excipients, such as binding agents, fillers, lubricants, disintegrants, and/or wetting agents. In some embodiments, a capsule, tablet, or soft gel form of the sleep aid compositions provided herein further includes one or more of microcrystalline cellulose, stearic acid, silicon dioxide, hydroxypropyl methylcellulose, sodium alginate, magnesium stearate, calcium carbonate, calcium sulfate, magnesium carbonate, cellulose gum, isomalt, polyethylene glycol, glycerin, hydroxypropyl cellulose, carnauba wax, and a flavorant.

In some embodiments, the capsule, tablet, or soft gel form of the sleep aid compositions provided herein do not include titanium dioxide. Titanium dioxide is a widely used food additive that can act as a whitening agent and stabilizer in dietary supplements. However, there is growing concern among consumers and researchers about the safety of this additive, and its potential negative impact on the gut microbiome, inflammation, and brain health.

In some embodiments, a tablet of a sleep aid composition provided herein can have a coating (e.g., a polymer or polysaccharide-based coating with or without plasticizers and/or pigments). Such coatings can include, for example, one or more of a film-forming polymer, a colorant, and a flavorant. Non-limiting examples of a flavorant include vanillin, vanilla extract, artificial vanilla flavorant, and natural caramel. In some embodiments, a sleep aid composition can have a vanilla or vanilla caramel flavor profile. In some embodiments, a coating of a tablet of a sleep aid composition provided herein includes one or more of: hydroxypropyl methyl cellulose, sodium alginate, calcium carbonate, calcium sulfate, magnesium carbonate, cellulose gum, isomalt, polyethylene glycol, glycerin, hydroxypropyl cellulose, and carnauba wax.

In some embodiments, a capsule, tablet, or soft gel form of the sleep aid compositions provided herein can have a unit dosage equal to the daily desired dosage for a particular mammal. For example, if it is desired to administer 100 mg of a particular agent to a mammal, each tablet can include about 100 mg in weight of that agent. As used herein, mammals generally refer to humans, but also can include domesticated mammals (e.g., dogs, cats, and livestock such as cows, horses, pigs, or sheep). The dosages of a particular sleep aid composition provided herein will depend on many factors, including the general health of a mammal. In some cases, a total daily dose (e.g., a full serving) can be prepared and administered in the form of one or more dosage units (e.g., one tablet, capsule, or soft gel, two tablets, capsules, or soft gels, three tablets, capsules, or soft gels, four tablets, capsules or soft gels, five tablets, capsules, or soft gels, or six tablets, capsules, or soft gels). For instance, in some cases, one tablet, one capsule, or one soft gel can include a full serving (e.g., daily dosage) of the composition. In some cases, two tablets, two capsules, or two soft gels in combination can include a full serving (e.g., daily dosage) of the composition. In some cases, three tablets, three capsules, or three soft gels in combination can include a full serving (e.g., daily dosage) of the composition.

In some embodiments, a sleep aid composition provided herein can be substantially free of artificial colors, artificial flavors, and chemical preservatives.

In some embodiments, a sleep aid composition provided herein can be a composition that includes ingredients as shown in Table 3.

TABLE 3

| Ingredient | Amount (mg) | Amount (% w/w) |
|---|---|---|
| GABA | 300 | 25 |
| L-Theanine | 200 | 17 |
| Ashwagandha extract | 128 | 11 |
| Melatonin | 5 | 0.4 |
| Binding agent(s), | 567 | 46.6 |
| filler(s), lubricant(s), | | |
| disintegrant(s), colorant(s), | | |
| and flavorant(s) | | |

In some embodiments, the sleep aid composition is formulated for both rapid and sustained release of the melatonin. In some embodiments, about 40% w/w of the melatonin is released within a first period of time (e.g., about 1 hour) and about 60% w/w of the melatonin is released over a second period of time (e.g., about 60% w/w of the melatonin is released over about 7 hours following the first period of time).

In some embodiments, the ashwagandha extract contains at least about 5% withanolides. In some embodiments, the ashwagandha extract contains about 5% withanolides. In some embodiments, the sleep aid composition contains about 6.25 mg withanolides.

### Methods

The sleep aid compositions provided herein can be useful for one or more of increasing relaxation, feeling relaxed more quickly, reducing stress, falling asleep quickly, and staying asleep. For example, a mammal may feel more relaxed, may feel relaxed more quickly, may feel less stress, may fall asleep faster (e.g., may have decreased sleep latency), and/or may stay asleep longer (e.g., may have increased sleep duration) after consuming a sleep aid composition provided herein as compared to a night where a sleep aid composition provided herein was not consumed.

Provided herein are methods of increasing relaxation in a mammal, comprising administering to the mammal any of the compositions described herein. In some embodiments, provided herein are methods of reducing stress in a mammal, comprising administering to the mammal any of the compositions described herein.

Also provided herein are methods of improving sleep quality in a mammal, comprising administering to the mammal any of the compositions described herein. For example, improving sleep quality can include decreasing sleep latency and/or increasing sleep duration. In some embodiments, provided herein are methods of decreasing sleep latency in a mammal, comprising administering to the mammal any of the compositions described herein. In some embodiments, provided herein are methods of increasing sleep duration in a mammal, comprising administering to the mammal any of the compositions described herein.

Relaxation, stress, and sleep (e.g., sleep duration and/or latency) can each be examined using any appropriate method(s). For example, sleep can be monitored using one or more of a sleep diary, a sleep questionnaire, a sleep tracker, and a polysomnography (PSG). A sleep diary can involve a mammal (e.g., a human) recording details such as their sleep habits, perceived sleep quality, and incidents of awakening during the night. Non-limiting examples of sleep questionnaires include the Pittsburgh Sleep Quality Index (PSQI) or the Epworth Sleepiness Scale. Non-limiting examples of a wearable sleep tracker include devices such as smartwatches that can monitor sleep stages, movements, and/or wakefulness throughout the night. A PSG is a study that can include recording brain waves, blood oxygen levels, heart rate, and breathing during sleep. A PSG can also measure eye and leg movements.

Further non-limiting examples of methods that can be used to examine relaxation, stress, and/or sleep include tests for anxiety (e.g., the Hamilton Anxiety Rating Scale (HAM-A) or the State-Trait Anxiety Inventory (STAI)); subjective ratings of pressure in response to standardized mental stress task-induced psychological stress; tests for mood (e.g., the Profile of Mood States (POMS)); and tests for stress (e.g., the Trier Social Stress Test (TSST)). See, e.g., Shinjyo et al. J. of Evidence-Based Integrative Med., 25:1-31 (2020).

In some embodiments, a sleep aid composition provided herein can be consumed by a mammal about 45 minutes to about 1.25 hours prior to a desirable time to fall asleep (e.g., bedtime). In some embodiments, a sleep aid composition provided herein can be consumed by a mammal about 1 hour prior to a desirable time to fall asleep (e.g., bedtime). In some embodiments, one serving of a sleep aid composition provided herein is consumed per day.

### EXAMPLES

### Example 1. Exemplary Dietary Compositions

A sleep aid composition was produced having the ingredients shown in Table 4.

TABLE 4

| Ingredient | Amount (mg) | Amount (% w/w) |
|---|---|---|
| GABA | 300 | 25 |
| L-Theanine | 200 | 17 |
| Ashwagandha extract | 128 | 11 |
| Melatonin | 5 | 0.4 |
| Binding agent(s), | 567 | 46.6 |
| filler(s), lubricant(s), | | |
| disintegrant(s), colorant(s), | | |
| and flavorant(s) | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A sleep aid composition comprising:
melatonin;
L-theanine present in an amount of about 150 mg to about 500 mg per serving;
GABA (gamma-aminobutyric acid) present in an amount of about 150 mg to about 450 mg per serving; and
an ashwagandha extract.

2. The sleep aid composition of claim 1, wherein the melatonin is present in an amount of about 0.5 mg to about 15 mg per serving.

3. (canceled)

4. (canceled)

5. The sleep aid composition of claim 1, wherein the sleep aid composition is formulated for rapid release and sustained release of the melatonin.

6. The sleep aid composition of claim 5, wherein about 20% to about 60% w/w of the melatonin is released within a first period of time.

7. (canceled)

8. The sleep aid composition of claim 6, wherein the first period of time is about 30 minutes to about 1 hour.

9. (canceled)

10. The sleep aid composition of claim 6, wherein about 40% to about 80% w/w of the melatonin is released over a second period of time following the first period of time.

11. (canceled)

12. The sleep aid composition of claim 10, wherein the second period of time is about 6 hours to about 8 hours.

13. (canceled)

14. The sleep aid composition of claim 1, wherein the L-theanine is present in an amount of about 100 mg to about 300 mg per serving.

15. (canceled)

16. The sleep aid composition of claim 1, wherein the GABA is present in an amount of about 200 mg to about 400 mg per serving.

17. (canceled)

18. The sleep aid composition of claim 1, wherein the ashwagandha extract is present in an amount of about 5 mg to about 600 mg per serving.

19. (canceled)

20. (canceled)

21. The sleep aid composition of claim 1, wherein the ashwagandha extract contains one or more withanolides.

22. The sleep aid composition of claim 21, wherein the one or more withanolides are present in an amount of about 1% to about 12% w/w of the ashwagandha extract.

23. (canceled)

24. The sleep aid composition of claim 1, wherein the sleep aid composition further comprises a valerian root extract.

25. The sleep aid composition of claim 1, wherein the sleep aid composition is formulated as a tablet, a capsule, or a soft gel.

26. (canceled)

27. The sleep aid composition of claim 25, wherein one tablet, capsule, or soft gel comprises a full serving of the composition.

28. A method of increasing relaxation in a mammal, the method comprising administering to the mammal a composition of claim 1.

29. A method of reducing stress in a mammal, the method comprising administering to the mammal a composition of claim 1.

30. A method of improving sleep quality in a mammal, the method comprising administering to the mammal a composition of claim 1.

31. A method of decreasing sleep latency in a mammal, the method comprising administering to the mammal a composition of claim 1.

32. A method of increasing sleep duration, the method comprising administering to the mammal a composition of claim 1.
